(19) United States
(12) Patent Application Publication
(10) Pub. No.: US 2019/0350821 A1
(43) Pub. Date: Nov. 21, 2019

(54) SURFACTANT-FREE EMULSION COMPOSITION

(71) Applicant: GREENVINES BIOTECH CO., LTD., Taipei City (TW)

(72) Inventors: Yi-Wen LIAO, Taipei City (TW); Jie LI, Taipei City (TW)

(21) Appl. No.: 16/401,284

(22) Filed: May 2, 2019

(30) Foreign Application Priority Data
May 17, 2018 (TW) 107116793

(51) Int. Cl. A61K 8/06; A61K 8/99; A61K 8/73; A61K 8/37; A61K 8/92; A61K 8/34; A61K 8/365

(52) U.S. Cl. CPC A61K 8/062; A61K 8/99; A61K 8/73; A61K 8/37; A61K 2800/33; A61K 8/345; A61K 8/365; A61K 2800/10; A61K 8/922

(57) ABSTRACT

A surfactant-free emulsion composition includes water, 18 wt % to 25 wt % of an oil component, and 0.35 wt % to 0.8 wt % of a gelling agent, based on. 100 wt % of the emulsion composition.

### CROSS-REFERENCE TO RELATED APPLICATION

This application claims priority of Taiwanese Patent. Application No. 107116793, filed on May 17, 2018.

### FIELD

The disclosure relates to an emulsion composition and more particularly to a surfactant-free emulsion composition.

### BACKGROUND

A conventional emulsion for cosmetics products on the market usually has increased oil component content so as to enhance moisture-retaining property, and a surfactant is necessarily added to avoid separation of the emulsion into oil and aqueous layers. However, the surfactant increases the stickiness feeling of the emulsion during use and might cause skin irritation. In addition, such conventional emulsion containing a surfactant might incur a higher manufacturing cost. Therefore, it is promising to develop an emulsion that does not contain a surfactant but can maintain a high amount of an oil component (i.e., can have a superior moisture-retaining property).

### SUMMARY

Therefore, an object of the present disclosure is to provide an emulsion composition which can alleviate at least one of the drawbacks associated with the prior art.

According to the present disclosure, the emulsion composition includes water, 18 wt % to 25 wt % of an oil component, and 0.35 wt. % to 0.8 wt % of a gelling agent, based on 100 wt % of the emulsion composition, and is free from a surfactant.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it should be clearly understood that the word “comprising” means “including but not limited to”, and that the word “comprise” has a corresponding meaning.

Unless otherwise defined, all technical and scientific terms used herein have the meaning as commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides an emulsion composition that includes water, 18 wt % to 25 wt % of an oil component, and 0.35 wt % to 0.8 wt % of a gelling agent, based on 100 wt % of the emulsion composition. The emulsion composition is free from a surfactant.

It should be noted that the particular range of the gelling agent content renders the surfactant-free emulsion composition of the present disclosure advantageous. Specifically, when the gelling agent is reduced to an amount of less than 0.35 wt %, the emulsion composition having such amount might become less stable, that is, the emulsion composition, even if being surfactant-free, is prone to separation into an oil layer and an aqueous layer. On the other hand, when the gelling agent is increased to an amount of more than 0.8 wt %, the emulsion composition having such amount may have increased viscosity, which might cause stickiness feeling and reduce the refreshing feeling when the emulsion composition is applied onto the skin of a subject, and which hinder further applications (for example, manufacture of the emulsion composition as a spray, etc.) Therefore, the control of the gelling agent content within the range of 0.35 wt % to 0.8 wt % can increase the stability of the emulsion composition of this disclosure (i.e., can prevent oil/water separation) and maintain the viscosity thereof at a satisfactorily low level.

As used herein, the term “gelling agent” refers to a component that can be partially dissolved or suspended in water to form a water-based gel or a viscous solution. Examples of the gelling agent suitable for use in this (disclosure may include, but are not limited to, xanthan gum, gellan gum, sclerotium gum, carbomer, guar gum, cellulose, and combinations thereof. In certain embodiments, the gelling agent is a combination of xanthan gum and gellan gum. In other embodiments, the gelling agent is a combination of gellan gum and sclerotium gum.

When the gelling agent is a combination of xanthan gum and gellan gum, the oil component may be present in an amount ranging from 18 wt % to 20 wt % and the gelling agent may be present in an amount ranging from 0.35 wt % to 0.55 wt %, based on 100 wt % of the emulsion composition. When the gel ling agent is a combination of gellan gum and sclerotium gum, the oil component may be present in an amount ranging from 23 wt % to 25 wt % and the gelling agent may be present in an amount ranging from 0.6 wt % and 0.8 wt %, based on 100 wt % of the emulsion composition.

As used herein, the term “oil component” refers to any water immiscible (substantially insoluble in water) component that is conventionally referred to as an oil and that is suitably used in the preparation of an emulsion.

Examples of the oil component suitable for use in this disclosure may include, but are not limited to, a medium-chain triglyceride, dicaprylyl carbonate, isononyl isononanoate, a plant squalane, dicaprylyl ether, and combinations thereof.

As used herein, the term “medium-chain triglyceride (MCI)” shall refer to a triglyceride containing one or more fatty acids, each having a medium-size chain length of about 8 to about 10 carbon atoms or mixtures thereof (i.e., medium-chain fatty acids (MCFAs)). The medium chain fatty acids can be either saturated or unsaturated, but are preferably saturated.

Examples of the MCI may include, but are not limited to, caprylic/capric triglyceride, caprylic triglyceride, capric triglyceride, and combinations thereof. In certain embodiments, the MCI is caprylic/capric triglyceride.

As used herein, the term “caprylic/capric triglyceride” shall encompass the triglyceride wherein the glycerol is esterified with caprylic and capric fatty acids (also termed “glyceryl caprylate caprate”), as well as a combination of glyceryl tricaprylate, glyceryl tricaprate, and glycerol caprylate caprate.

Examples of the plant oil suitable for use in this disclosure may include, but are not limited to, Helianthus annuus seed oil (i.e., sunflower seed oil), Simmondsia chinensis seed oil (i.e., jojoba seed oil), Camellia oleifera seed oil, Rosa canina seed oil, Sambucus nigra seed oil (i.e., black elder seed oil), Raphanus sativus seed oil (i.e., radish seed oil), Prunus amygdalus dulcis oil (i.e., almond seed oil), and combinations thereof.

In certain embodiments, the emulsion composition further includes a preservative.

As used herein, the term “preservative” refers to any component that is commonly added into an emulsion composition to inhibit the growth of microorganisms.

Examples of the preservative suitable for use in this disclosure may include, but are not limited to, Lactobacillus/radish root ferment filtrate, levulinic acid, sodium levulinate, sodium citrate, glycerol and combinations thereof.

In certain embodiments, the preservative is present in an amount ranging from 4 wt % to 6 wt % based on 100 wt % of the emulsion composition.

This disclosure will be further described by way of the following examples. However, it should be understood that the following examples are solely intended for the purpose of illustration and should not be construed as limiting the disclosure in practice.

### EXAMPLES

### Example 1. Preparation of Surfactant-Free Emulsion Composition

The surfactant-free emulsion composition of Experimental Group 1 was prepared according to the following procedures and using the first formulation shown in Table 1 below. First, the gelling agent was mixed with water under stirring so as to obtain a first mixture. The oil component was homogeneously mixed, and then was mixed with the first mixture under stirring so as to obtain a second mixture. The preservative was homogeneously mixed, and then was mixed with the second mixture under stirring so as to obtain the surfactant-free emulsion composition of Experimental Group 1.

TABLE 1

| | | Amount |
|---|---|---|
| First formulation | | (wt %) |
| Water | | Balance |
| Gelling | Gellan gum | 0.2 |
| agent | Xanthan gum | 0.15 |
| Oil | Caprylic/capric triglyceride (Myritol ® 318 | 18 |
| component | available from BASF, France S.A.S) | |
| | Dicaprylyl carbonate | |
| | Helianthus annuus seed oil | |
| | Camellia oleifera seed oil | |
| | Rosa canina fruit oil | |
| | Simmondsia chinensis seed oil | |
| | Sambucus nigra seed oil | |
| | Raphanus sativus seed oil | |
| | Prunus amygdalus dulcis oil | |
| Preservative | Lactobacillus/radish root ferment filtrate | 4 |
| | (Leucidal ® Liquid AE LFHC available from | |
| | Active Micro Technologies LLC, USA) | |
| | Glycerin, levulinic acid, and sodium | 1 |
| | levulinate (Dermosoft ® 700B available from | |
| | Evonik Dr. Straetmans GmbH, Germany) | |
| | Sodium citrate | 0.03 |

The surfactant-free emulsion composition of Experimental Group 2 was prepared according to the procedures similar to those of Experimental Group 1 as mentioned above, except for using the second formulation shown in Table 2 below.

TABLE 2

| | | Amount |
|---|---|---|
| Second formulation | | (wt %) |
| Water | | Balance |
| Gelling | Gellan gum | 0.15 |
| agent | Sclerotium gum | 0.65 |
| Oil | Caprylic/capric triglyceride (Myritol ® 318 | 25 |
| component | available from BASF, France S.A.S) | |
| | Dicaprylyl carbonate | |
| | Helianthus annuus seed oil | |
| | Camellia oleifera seed oil | |
| | Rosa canina fruit oil | |
| | Simmondsia chinensis seed oil | |
| | Sambucus nigra seed oil | |
| | Raphanus sativus seed oil | |
| | Prunus amygdalus dulcis oil | |
| Preservative | Lactobacillus/radish root ferment filtrate | 4 |
| | (Leucidal ® Liquid AE LFHC available from | |
| | Active Micro Technologies LLC, USA) | |
| | Glycerin, levulinic acid, and sodium | 1 |
| | levulinate (Dermosoft ® 700B available from | |
| | Evonik Dr. Straetmans GmbH, Germany) | |
| | Sodium citrate | 0.03 |

### Example 2. Stability Test for Surfactant-Free Emulsion Composition

To determine the stability of the surfactant-free emulsion compositions of Experimental Groups 1 and 2 prepared in Example 1 with respect to high temperature and centrifugation treatments, the following experiments were conducted.

### A. High Temperature Treatment

The surfactant-free emulsion compositions of Experimental Groups 1 and 2 were placed in an oven at 50° C. for 12 weeks. It was found that after the high temperature treatment, both of the surfactant-free emulsion compositions of Experimental Groups 1 and 2 did not separate into an oil layer and an aqueous layer.

### B. Centrifugation Treatment

The surfactant-free emulsion compositions of Experimental Groups and 2 were centrifuged at a speed of 3000 rpm for 20 minutes. It was found that after the centrifugation treatment, the surfactant-free emulsion compositions of Experimental Groups 1 and 2 did not separate into an oil layer and an aqueous layer.

The results reveal that even after high temperature and centrifugation treatments, the emulsion composition of this disclosure, which is free from any surfactant and includes 0.35 wt % to 0.8 wt % of a gelling agent and a high amount (ranging from 18 wt % to 25 wt %) of an oil component, can have excellent stability and does not separate into an oil layer and an aqueous layer. The applicant therefore expects that the surfactant-free emulsion composition of this disclosure will not separate into oil and aqueous layers within 3 years after manufacture.

In summary, by including 0.35 wt % to 0.8 wt % of a gelling agent, the emulsion composition or this disclosure can maintain a high amount. (ranging from 18 wt % to 25 wt %) of an oil component (i.e., can have a superior moisture-retaining property) while exhibiting a long-lasting satisfactory stability in the absence of a surfactant.

All patents and literature references cited in the present specification as well as the references described therein, are hereby incorporated by reference in their entirety. In case of conflict, the present description, including definitions, will prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. An emulsion composition, comprising: water, 18 wt % to 25 wt % of an oil component, and 0.35 wt % to 0.8 wt % of a gelling agent, based on 100 wt % of said emulsion composition, said emulsion composition being free from a surfactant.

2. The emulsion composition as claimed in claim 1, wherein said gelling agent is selected from the group consisting of xanthan gum, gellan gum, sclerotium gum, carbomer, guar gum, cellulose, and combinations thereof.

3. The emulsion composition as claimed in claim 2, wherein said gelling agent is a combination of xanthan gum and gellan gum.

4. The emulsion composition as claimed in claim 2, wherein said gelling agent is a combination of gellan gum and sclerotium gum.

5. The emulsion composition as claimed in claim 1, wherein said oil component is selected from the group consisting of a medium-chain triglyceride, dicaprylyl carbonate, isononyl isononanoate, a plant oil, squalane, dicaprylyl ether, and combinations thereof.

6. The emulsion composition as claimed in claim 5, wherein said medium-chain triglycerides are selected from the group consisting of caprylic/capric triglyceride, caprylic triglyceride, capric triglyceride, and combinations thereof.

7. The emulsion composition as claimed in claim 5, wherein said plant oil is selected from the group consisting of Helianthus annuus seed oil, Simmondsia chinensis seed oil, Camellia oleifera seed oil, Rosa canina seed oil, Sambucus nigra seed oil, Raphanus sativus seed oil, Prunus amygdalus dulcis oil, and combinations thereof.

8. The emulsion composition as claimed in claim 1, further comprising a preservative.

9. The emulsion composition as claimed in claim 8, wherein said preservative is present in an amount ranging from 4 wt % to 6 wt % based on 100 wt % of said emulsion composition.

10. The emulsion as claimed in claim 8, wherein said preservative is selected from the group consisting of Lactobacillus/radish root ferment filtrate, levulinic acid, sodium levulinate, sodium citrate, glycerol, and combinations thereof.
